(19) United States
(12) Patent Application Publication
(10) Pub. No.: US 2024/0226495 A1
(43) Pub. Date: Jul. 11, 2024

(54) TRANQUILIZING SYSTEM USING HEART RATE VARIABILITY TO SIMULATE PRESENCE, SOOTHE ANXIETY, AND PROMOTE SYMPATHOVAGAL BALANCE

(71) Applicants: Chet Anthony, Ormond Beach, FL (US); Amy Louise Anthony, Ormond Beach, FL (US)

(72) Inventors: Chet Anthony, Ormond Beach, FL (US); Amy Louise Anthony, Ormond Beach, FL (US)

(21) Appl. No.: 17/531,762

(22) Filed: Nov. 21, 2021

(51) Int. Cl. A61M 21/00

(52) U.S. Cl. CPC A61M 21/00; A61M 2021/0022; A61M 2021/0027; A61M 2021/0061

(57) ABSTRACT

A heart rate variability collection and transmission system for simulation of presence, soothing and vasovagal balance includes a variety of possible means of collection, storage, and transmissibility. Each means of collection senses subtle variation of heart rate. Storage is via a computerized data file and consists of time intervals from the collected source. Time intervals are used as instructions to perform an action such as activating a haptic motor, audio file or transducer at that collected rate. Transmission of action is wireless or direct (wired). The motor, transducer, or audio-file is activated at the collected rate. The effect is achieved.

### TECHNICAL FIELD

This disclosure relates to a heart rate variability (HRV) collection and transmission system that can be controlled by an application that runs on a smartphone, ECG, or any smart device. The application collects fluctuations in heartbeat, records them in a file, and reproduces the information in the form of sound file (in many forms or as heartbeat sound) or tactile response, thus simulating the sensations experienced when lying in juxtaposition with the recorded source (for example: a mother. The potential to ensure calmness, soothe anxiety, sympathovagal balance or even neurological development is thus induced.

### BACKGROUND

A general feeling of wellness and calm is a precious asset. It is accepted that the heart rate variability, which is the physiological phenomenon in the time interval between heartbeats, can be a measure of general health (in the form of Autonomic Balance) and measure of stress.

Autonomic balance (also known as sympathovagal balance or SB) can reduce mortality, morbidity, and improve quality of life. Cardiac autonomic balance (CAB), also known as the relative influences of the sympathetic (SNS) and the parasympathetic nervous systems on the heart at rest, is an established biomarker of physical and mental health. This can be measured in samples of HRV.

In some situations such as during separation, when experiencing social phobias, and medical situations (such as when experiencing palpitations, high blood pressure, irritable bowel, anxiety, insomnia), it may be desirable or beneficial to expose animals and or individuals to healthy sources of HRV (via audio and tactile experiences). This can help promote wellness, growth, and soothing. Some situations where this could be applicable are separation from a mother and a child (NICU, daycare, etc.) or soothing animals with separation anxiety, PTSD, or severe negative reactions to storms, fireworks, etc. Adults with anxiety and other health issues or stress can benefit from the soothing effects of HRV.

### SUMMARY

Against this background it would be desirable to have a system to collect healthy HRV, store it as a data file, and reproduce it in a stimulating device. The information could then be used as tactile and/or auditory input to the recipient.

The assembly in several possible embodiments uses personalized recorded files that are optimally created from a recipient, preferably in a joyous emotional state, which maximizes variability. A phone application, ECG, or smart device (app) subsequently communicates with a speaker, haptic device, or any tactile device, thus simulating a heartbeat. The communication can be to the phone's internal haptic motor (or transducer) or to any wireless connected device containing a haptic motor or device capable of producing a tactile sensation. Multiple devices may all be connected and synched to help multiple recipients at once or in real time.

Among the features of the disclosed system and method are:
1. Ability to record heart rate intervals.
2. Ability to play a sound file (such as a heartbeat sound)/produce a pressure wave with collected variable heart rate intervals.
3. Ability to influence haptic motors or any connected tactile device with collected variable heart rate intervals.
4. Wired or wireless connectivity (such as Bluetooth, Wi-Fi, etc.).
5. Reproduce variable heart rates (HRV) in the form of a tactile response specific to the operator.

### BRIEF DESCRIPTION OF THE DRAWINGS

FIG. 1 is a representative cross section of the collection of HRV via camera, LED, or ECG, etc.

FIG. 2 is a flow chart illustrating the steps of transmission of the HRV file directly or wirelessly to a tactile instrument.

### DETAILED DESCRIPTION

As required, detailed embodiments of the present invention are disclosed herein, however, it is to be understood that the disclosed embodiments are merely exemplary of the invention that may be embodied in various and alternative forms. The figures are not necessarily to scale, some features may be exaggerated or minimize to show details of particular components. Therefore, specific structural and functional details disclosed herein are not to be interpreted as limiting, but merely as a representative basis for teaching one skilled in the art to variously employ the present invention.

Specific details disclosed here are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below.

The embodiment of the disclosed system and the related steps involve structural and electronic features that lend for human use. However, it should be appreciated that as the translatory component is adjusted to include multiple physical devices such as watches, wraps, stretchy bracelets, swaddles, pads, clothing, animal collars, animal beds, etc. and it should be appreciated that it can be used for all mammals. If the signal is transmitted via a transducer/speaker it should be appreciated that it also holds useful and invitation for all species with auditory capabilities

Soothing effects, reduction of anxiety and palpitations, autonomic balance, and neurocognitive benefit are induced by mimicking the experience of being in juxtaposition with a healthy autonomic source of HRV such as a mother or loved one but not necessarily with that limitation.

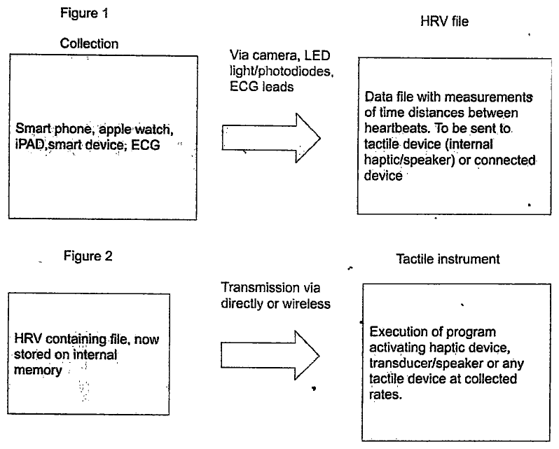

A representative assembly of possible configurations is shown in FIG. 1. The phone, watch, tablet, or other smart device included has the ability to collect pulses (heartbeats) through a camera (which tracks color change through an appendage such as a fingertip) or LED lights paired with photodiodes. The portable ECG collects electrical activity from the heart from at least one lead. Each collection is a heart rate and thus a representation of heart rate variability (preferably collected during a time of positive emotional experience or state). The collection of data will have variable distances between each successive heartbeat and thus by definition be “variable”. The data will be stored as a file (in internal memory) or internally on an ECG computer.

The data is then distributed in FIG. 2, $o several possible devices or if the collection device has the ability to produce a tactile response, can be done directly.

The tranquilizing endpoint (playback) can occur in any embodiment that has the ability to produce a tactile response. Within a phone, the internal haptics can be used and synchronized to files with HRV data files (time intervals between collected heartbeats). The phone's speaker can also be used to play a prerecorded heartbeat with the same data (same rate).

Step (1) Contemplates the various means of collecting HRV data. A smartphone, ECG, iPad, or other smart device aid in the collection of intervals between heartbeats over a defined period of time (or continuously if needed). The collection occurs via a phone's camera, LED lights and photodiode, or lead usage (as in the case of ECG).

Step (2) contemplates storage and processing. The data (now stored on the phone or devices internal memory) collected becomes a series of time intervals by which to perform an action (in this case, the action being to send a command to activate haptics, transducers, or any tactile action device) via a connection that is direct(wired) or via Bluetooth, Wi-Fi, Li Fi, Zigbee, NFC, 5G, etc.

Step (3) Contemplates transmission directed or connected.

Step (4) Contemplates playback/production. In a phone or smart device with haptics, haptic motors are activated at the same intervals recorded in Step (1). When an external device is used (for playback) such as, but not limited to a band, bed, or mat embedded with haptics, a wireless connection is established, and a transmission is sent to activate haptics or any tactile sensation at the collected time intervals from Step (1). The same process would occur for an ECG with internal haptics and would work for an external device via the same playback process.

At contemplated endpoints there are tactile or auditory (if using transducer/speaker) inputs at variable frequencies that are directly collected from the Step (1) source.

While embodiments are described above, it is not intended that these embodiment describe all possible forms of the invention. Rather, the words used in the specification are words of description rather than limitation, and it is understood that various changes may be made without departing from the spirit and scope of the invention. Additionally, the features of various impending embodiments may be combined to form further embodiments of the invention.

1. HRV collection system and/or application containing a phone's camera, ECG, pulse detection device or any smart device. This device collects time intervals between successive heartbeats or time intervals between characteristic electric changes on an ECG (such as R-R intervals). These time intervals are converted to a data file. This data file can be stored, then subsequently converted to a sound file, and played through a transducer/speaker or converted to any device that can produce a tactile response (such as a haptic motor).

2. The time signature (HRV) data will be unique to the collected source and synonymous with the collected source's mood (Autonomic Status).

3. The subsequent transmission to the transformer/speaker will create a sound/pressure wave. The transmission to a haptic motor or other will produce a tactile response. The presence of the healthy recorded entity will thus be simulated. Repeated exposure to a healthy source example of sympathovagal balance (a healthy HRV state) will occur. This will in turn promote soothing (decrease stress), promote autonomic balance, and can help treat all disorders with autonomic imbalance (irritable bowel disease, mood disorder, insomnia, anxiety, etc.). And decrease the likelihood of palpitations or arrhythmias (which can occur during stress).
